SEQUENCE LISTING (4 sequences)

SEQ ID NO: 1 - DNA, 23 bp, Artificial sequence
  Synthetic oligonucleotide
ggauacagcuggagucagudtdt

SEQ ID NO: 2 - DNA, 23 bp, Artificial sequence
  Synthetic oilgonucleotide
acugacuccagcuguauccdtdt

SEQ ID NO: 3 - DNA, 23 bp, Artificial sequence
  Synthetic oligonucleotide
cagcuguauuccauuacaudtdt

SEQ ID NO: 4 - DNA, 23 bp, Artificial sequence
  Synthetic oligonucleotide
aucuaauggaauacagcugdtdt

(19) United States
(12) Patent Application Publication
(10) Pub. No.: US 2019/0183948 A1
(43) Pub. Date: Jun. 20, 2019

(54) USE OF BCL-XL INHIBITOR AND ONCOLYTIC VIRUS IN PREPARATION OF ANTITUMOR DRUG

(71) Applicant: GUANGZHOU VIROTECH PHARMACEUTICAL CO., LTD., Guangzhou, Guangdong (CN)

(72) Inventors: Guangmei YAN, Guangzhou (CN); Yaqian TAN, Guangzhou (CN); Yuan LIN, Guangzhou (CN); Haipeng ZHANG, Guangzhou (CN); Suizhen LIN, Guangzhou (CN); Shoufang GONG, Guangzhou (CN); Jun HU, Guangzhou (CN); Xiao XIAO, Guangzhou (CN); Kai LI, Guangzhou (CN); Jiankai LIANG, Guangzhou (CN); Jing CAI, Guangzhou (CN); Wenbo ZHU, Guangzhou (CN); Wei YIN, Guangzhou (CN)

(73) Assignee: GUANGZHOU VIROTECH PHARMACEUTICAL CO., LTD., Guangzhou, Guangdong (CN)

(21) Appl. No.: 16/325,867

(22) PCT Filed: Aug. 18, 2017
(86) PCT No.: PCT/CN2017/097971

§ 371 Date: Feb. 15, 2019

(30) Foreign Application Priority Data
Aug. 18, 2016 (CN) 201610688096.4

(51) Int. Cl. A61K 35/768; A61K 31/11; A61K 31/352; A61K 31/428; A61K 31/4725; A61K 31/5377; A61K 31/635; A61K 31/7105; A61P 35/00

(52) U.S. Cl. CPC A61K 35/768; A61K 31/11; A61K 31/352; A61K 31/428; A61P 35/00; A61K 31/5377; A61K 31/635; A61K 31/7105; A61K 31/4725

(57) ABSTRACT

This invention belongs to the field of biomedicine and relates to use of Bcl-xL inhibitor and oncolytic virus in the preparation of an anti-tumor drug. The present invention firstly discovers that the Bcl-xL inhibitor can be used as an anti-tumor synergist for oncolytic virus. The present invention also relates to a pharmaceutical composition comprising Bcl-xL inhibitor and oncolytic virus, a pharmaceutical kit comprising Bcl-xL inhibitor and oncolytic virus, and use of Bcl-xL inhibitor and oncolytic virus for treating tumors, especially, tumors that are not sensitive to the oncolytic virus.

### BACKGROUND OF THE INVENTION

### Technical Field

The present disclosure relates to the field of biomedicine and relates to the combination of Bcl-xL inhibitor and oncolytic virus in the preparation of an anti-tumor drug.

### Background Art

Oncolytic virus refers to a class of replicatable viruses that infect and kill tumor cells in a targeted way and without affectint non-tumor cells. Oncolytic virotherapy, being an innovative strategy for a tumor targeted therapy, can use a natural or genetically modified virus (or combination thereof) to selectively infect tumor cells and replicates said virus in the tumor cells, so as to achieve the effect of dissolving and killing the tumor cells, preferably in a target way, and preferably with little or no damage to normal cells.

One example of an oncolytic virus is the M1 virus (Alphavirus M1) which belongs to Alphavirus sp., and can have potential application in the preparation of an anti-tumor drug. For example, Chinese invention patent application No. 201410425510.3 discloses that M1 virus could selectively cause the death of tumor cells without affecting the survival of normal cells. Nevertheless, different tumors show different sensitivity to M1 virus. For certain tumors, M1 virus, when being administered alone, does not exhibit a satisfactory oncolytic effect. According to the Chinese invention patent application No. 201410425510.3, incorporated herein by reference for its discussion of treatment of tumors with M1 virus and other viruses and for its discussion of different outcomes for different tumor types and treatment levels/methodologies, it is disclosed that M1 present different efficacy when treating different tumor type. When used for treating pancreatic cancer, nasopharyngeal carcinoma, prostate cancer and melanoma, it presents a most effective result. When used for treating colorectal cancer, liver cancer, bladder cancer and breast cancer, it is less effective than the previous mentioned type. When used for treating glioma, cervical cancer, lung cancer, it is even less effective. When used for treating gastric cancer, it presents a least effective result.

The screening of the compounds that increase therapeutic effect of oncolytic virus on tumor is underway to identify compounds that can increase the anti-tumor spectrum and anti-tumor strength of oncolytic virus. Chinese patent CN 201510990705.7 previously submitted by the inventor discloses that chrysophanol are used as anti-tumor synergists for M1 virus, and a combination of both could reduce the survival rate of tumor cells to 39.6%. Nevertheless, there is still much progress to be made in developing therapeutic combinations that include an oncolytic virus. The object of the present disclosure is to provide an anti-tumor synergist for oncolytic virus, such synergist could produce better anti-tumor effect.

### BRIEF SUMMARY OF THE INVENTION

Provided is a use of a Bcl-xL inhibitor in the preparation of an anti-tumor synergist for oncolytic virus.

Also provided is an anti-tumor pharmaceutical composition which enables oncolytic virus to exhibit better antitumor effect.

Also provided is a synergistic drug combination with oncolytic virus, and which is preferably directed to tumors that are not sensitive to oncolytic virus.

Also provided are methods of treatment of a solid or hematological (i.e. blood) tumor that include administration of the foregoing therapeutic combinations.

The aforementioned objects are achieved by the following technical solution.

After study, the inventors found that Bcl-xL inhibitor can enhance the oncolytic effect of oncolytic virus.

### BRIEF DESCRIPTION OF THE DRAWINGS

FIG. 1 shows that ABT-263 and M1 virus significantly enhance the pathological change of human liver cell strain in terms of morphology.

FIG. 2 shows that the combined treatment of ABT-263/ABT-737 and M1 virus significantly reduces the survival rate of human liver cancer cell strain.

FIG. 3 shows the study on the anti-tumor mechanism of the combination of Bcl-xL protein inhibitor and oncolytic virus.

FIG. 4 shows that the combined treatment of ABT-263 and M1 virus significantly inhibits the growth of transplantable tumor of human liver cancer cell strain.

### DESCRIPTION OF SYMBOLS IN THE FIGURES

ABT-263: ABT-263 treatment group; ABT-737: ABT-737 treatment group; M1+ABT-263: a combined treatment group using M1 virus and ABT-263; M1+ABT-737: a combined treatment group using M1 virus and ABT-737.

### DETAILED DESCRIPTION OF THE INVENTION

Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Use of words such as “approximate”, “about”, “substantially”, etc. modifies the value/description as would be understood by a person of skill in the art based upon the context and the parameter being described, and where further guidance is needed in order to be understood, a value of 5% can be applied. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.” Described percent sequence identity refers to the percentage of nucleic acid or amino acid residues within a given DNA, RNA or protein, respectively, that are identical to the reference sequence.

In case of conflict, the present specification, including explanations of terms, will control. In addition, all the materials, methods, and examples are illustrative and not intended to be limiting.

Anti-Tumor Combinations of a Bcl-xL Inhibitor and an Oncolytic Alphavirus

Said Bcl-xL inhibitor is a substance that inhibits the activity of Bcl-xL protein, a substance that degrades Bcl-xL protein, or a genetic tool that reduces the level of Bcl-xL protein.

The proteins expressed by Bcl-2 family genes are called Bcl-2 family proteins. Bcl-2 family members are evolutionarily related proteins, which control mitochondrial outer membrane permeabilization (MOMP). It is known that the Bcl-2 family consists of 25 genes, of which some members such as Bax, BAD, Bak and Bok can promote apoptosis, while some members such as bcl-2, Bcl-xL and Bcl-w can prevent apoptosis. Bcl-2 family protein is an important protein in the apoptosis pathway and plays an important role in tumorigenesis and metastasis. Since the Bcl-2 family proteins including bcl-2, Bcl-xL and Bcl-w are highly expressed in cancer cells, Bcl-2 family protein inhibitors can selectively produce anti-tumor effects in tumor cells.

Nevertheless, it is difficult to expect that all of a Bcl-2 family protein inhibitors can synergistically enhance the oncolytic effect of oncolytic virus.

As described herein, an interference fragments (siRNAs) of Bcl-xL and Bcl-w was used to inhibit the expression of the two genes, thereby reducing the expression of the corresponding proteins. The results showed that the interference of Bcl-xL or Bcl-w alone did not result in a pathological change of cell morphology, so as the non-interference group. Also, use of M1 virus alone did not cause pathological change of cell morphology. However, a combination of interference of Bcl-xL an application of M1 virus caused significant pathological change of cell morphology. On the other hand, the combination of interference of Bcl-w with application of M1 virus did not cause significant pathological change of cell morphology. In addition, when the inventors used the combination of the Bcl-2 inhibitor ABT-199 and oncolytic virus to treat tumor cells, no significant difference in the survival rate of the tumor cells was found, showing that inhibition of Bcl-2 cannot result in an increased oncolytic effect of M1 virus.

The inventors, therefore, speculated that the oncolytic effect of oncolytic virus can be significantly enhanced only by inhibiting Bcl-xL. To examine this speculation, the inventors used the Bcl-xl inhibiting active compound ABT-263 or ABT-737 in combination with oncolytic virus, in particular with M1 virus, to treat tumor cells, and found that ABT-263, ABT737 or a combination thereof could synergistically act with oncolytic virus to enhance the anti-tumor effect of the oncolytic virus.

ABT-263 (Navitoclax), one of the Bcl-2 family protein inhibitors, is an inhibitor of Bcl-xL, Bcl-2 and Bcl-w with Ki≤0.5 nM, ≤1 nM and ≤1 nM respectively, but binds weakly to Mcl-1 and A1.

ABT-737 is a BH3 mimetic inhibitor that acts on Bcl-xL, Bcl-2 and Bcl-w, with EC50 of 78.7 nM, 30.3 nM and 197.8 nM; but has no inhibitory effect on Mcl-1, Bcl-B and Bfl-1. ABT-199 (GDC-0199) is a potential selective inhibitor of Bcl-2 with Ki of 0.01 nM, and its inhibitory effect on Bcl-2 is more than 4,800 times than the inhibitory effect on Bcl-xL and Bcl-w, but has no activity on Mcl-1.

The present disclosure firstly found that the Bcl-xL inhibitor can be used as an anti-tumor synergist for oncolytic virus.

The present disclosure provides the use of Bcl-xL inhibitor in the preparation of an anti-tumor synergist for oncolytic virus.

The Bcl-xL inhibitor is a substance (e.g., a compound, an amino acid sequence or a nucleotide sequence) or a tool capable of knocking down or affecting the gene expression of Bcl-xL or reducing the protein amount or protein activity of Bcl-xL. Those skilled in the art could, among others, modify, replace and/or change the inhibiting compound, sequence or genetic tool. Provided that the resulting substance has the effect of inhibiting Bcl-xL, such substance belongs to the Bcl-xL inhibitor of the present invention, and belongs to homogenous replacement of the above substance, compound and tool.

Said Bcl-xL inhibitor can include (but is not limited to) (S)-Gossypol acetic acid (Formula 1), Apogossypol (Formula 2), A-1155463 (Formula 3), AT-101 (R-(−)-gossypol acetic acid (Formula 4), WEHI-539 and WEHI-539 hydrochloride (Formula 5), Gambogic Acid (Formula 6), A-1210477 (Formula 7), ABT-263 (Formula 8), ABT-737 (Formula 9) and other compounds that inhibit the activity of Bcl-xl protein. The compounds can be obtained through but not limited to chemical separation or synthesis per se or commercial purchase.

Other low molecular weight compounds that inhibit the activity of Bcl-xl protein, and which may be used as the Bcl-xL inhibitor herein, include BH3I (Formula 10), BH3I-1 (Formula 11), TW-37 (Formula 12), 2-methoxy-antimycin A3 (Formula 13), BI-97C1 (Formula 14), ABT-199 (Formula 15), BM-1197 (Formula 16), and A-1331852 (Formula 17). Additional Bcl-xL inhibitors useable herein are described in Hennessy (Bioorganic & Medicinal Chemistry Letters, 2016, 26: 2105-2114; incorporated by reference herein in its entirety).

In a preferred embodiment of the present invention, the Bcl-xL inhibitor can be ABT-263, ABT-737 or a combination thereof.

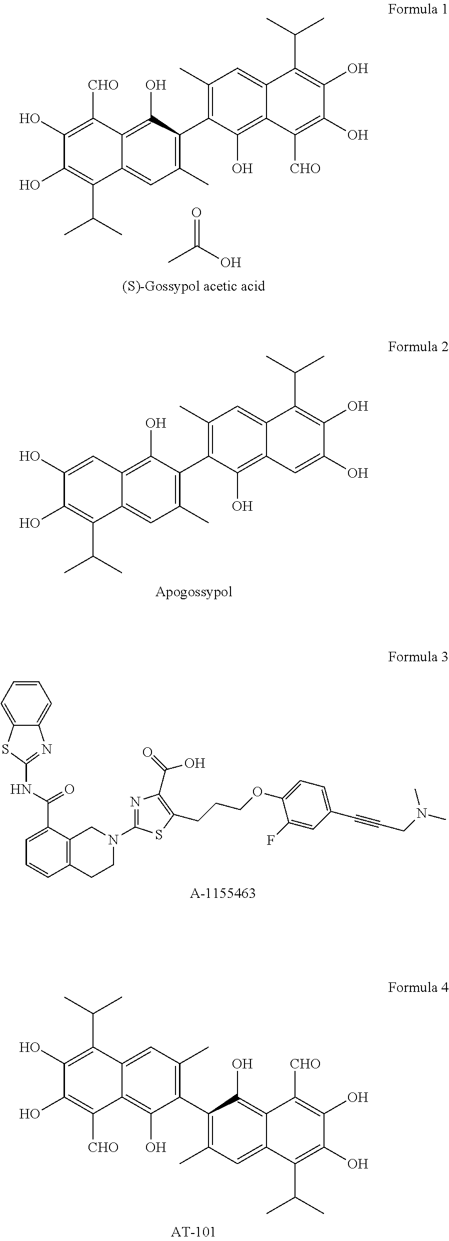

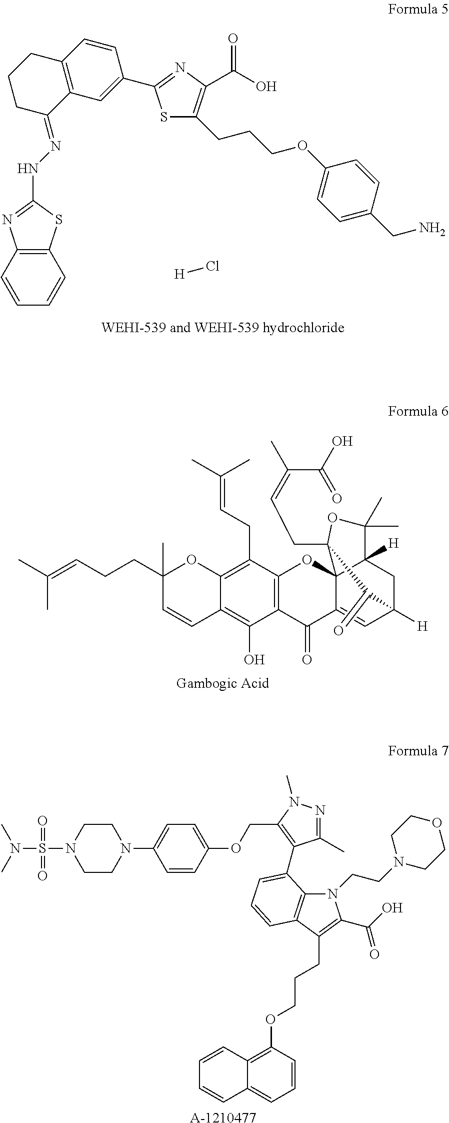

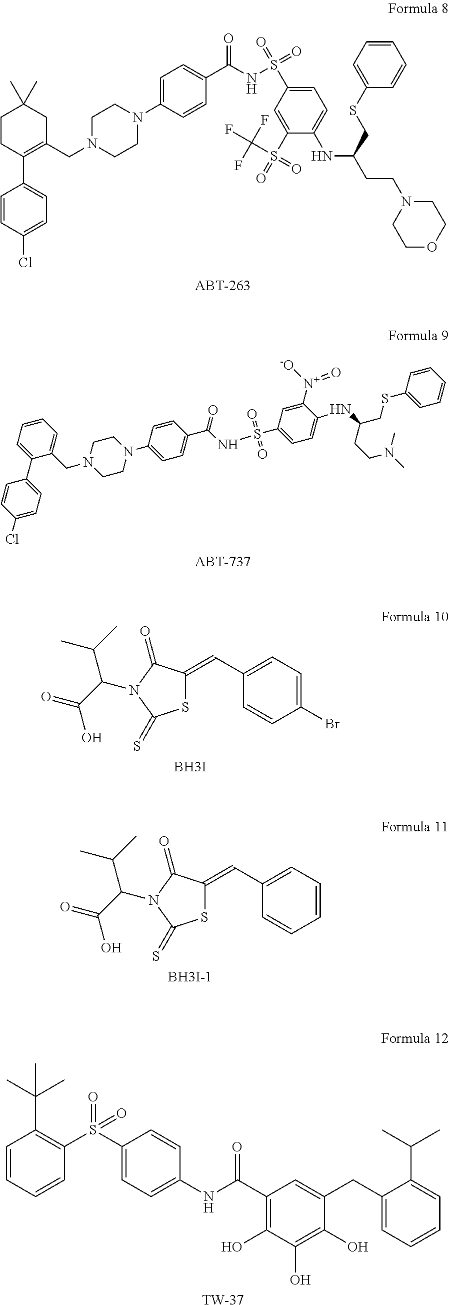

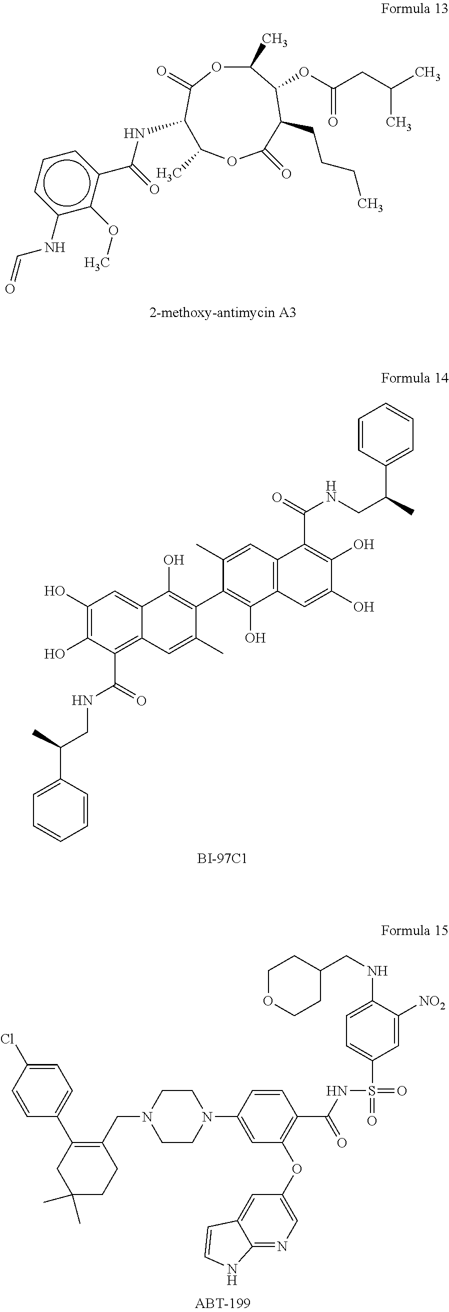

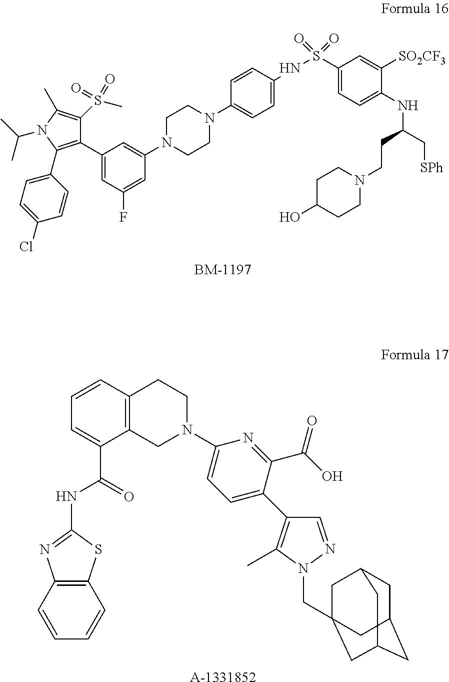

Bcl-xL inhibitor can also include a genetic-based tool for inhibiting the gene expression of Bcl-xl, including (but not limited to) RNA interference (RNAi), microRNA and gene editing or gene knockout material.

The Bcl-xL inhibitor also includes, for example, small inhibitory nucleic acid molecules, such as short interfering RNA (siRNA), double-stranded RNA (dsRNA), micro-RNA (miRNA), ribozyme, and short hairpin RNA (shRNA), that decrease or ablate expression of Bcl-xL.

Such small inhibitory nucleic acid molecules may comprise a first and a second strand that hybridize to each other to form one or more double-stranded regions, each strand being about 18 to about 28 nucleotides in length, about 18 to about 23 nucleotides in length, or about 18, 19, 20, 21 or 22 nucleotides in length. Alternatively, a single strand may contain regions therein capable of hybridizing to each other to form a double-stranded region, such as in shRNA molecules.

Such small inhibitory nucleic acid molecules may also comprise modified nucleotides, while maintaining an ability to reduce or ablate Bcl-xL expression. The modified nucleotides may be included to improve in vitro or in vivo characteristics, such as stability, activity, and/or bioavailability. For example, such small inhibitory nucleic acid molecules may comprise modified nucleotides as a percentage of the total number of nucleotides present in the siRNA molecule, such as at least about 5%, 10%, 15%, 20%, 25%, 30%, 35%, 40%, 45%, 50%, 55%, 60%, 65%, 70%, 75%, 80%, 85%, 90%, 95% or 100% modified nucleotides). Such modified nucleotides may comprise, for example, deoxynucleotides, 2′-O-methyl nucleotides, 2′-deoxy-2′-fluoro nucleotides, 4′-thionucleotides, locked nucleic acid (LNA) nucleotides, and/or 2′-O-methoxyethyl nucleotides. The small inhibitory nucleic acid molecules, such as siRNAs, may also contain a ‘5- and/or a 3’-cap structure, to prevent degradation by exonucleases.

In some embodiments, the Bcl-xL inhibitor is a nucleic acid that is at least 90%, at least 91%, at least 92%, at least 93%, at least 94%, at least 95%, at least 96%, at least 97%, at least 98%, or at least 99% identical to any of the nucleic acids set forth as SEQ ID Nos 1-4.

In some embodiments, the small inhibitory nucleic acid molecules comprise double-stranded nucleic acids containing blunt ends, or overhanging nucleotides. Other nucleotides present may comprise, for example, nucleotides that result in mismatches, bulges, loops, or wobble base pairs. The small inhibitory nucleic acid molecules may be formulated for administration, for example, by encapsulation in liposomes, or by incorporation into other vehicles, such as biodegradable polymers, hydrogels, or cyclodextrins.

In a preferred embodiment of the present invention, the Bcl-xL protein inhibitor is an interference RNA fragment of Bcl-xL, or an antibody against Bcl-xL. In some embodiments, the Bcl-xL inhibitor is effective against, designed to target, produced or raised using, or is specific for, Bcl-xL set forth in Genbank Accession No. Z23115 (amino acid sequence or nucleic acid sequence). In other embodiments, the Bcl-xL inhibitor is effective against, designed to target, produced or raised using, or is specific for, a variant of the Bcl-xL set forth in Genbank Accession No. Z23115. The variant Bcl-xL protein may have, for example, an amino acid sequence that is at least 80%, at least 85%, at least 90%, at least 91%, at least 92%, at least 93%, at least 94%, at least 95%, at least 96%, at least 97%, at least 98%, or at least 99%, identical to the Bcl-xL protein set forth in Genbank Accession No. Z23115.

In some embodiments, the Bcl-xL inhibitor is an antibody. The antibody may be a monoclonal antibody, a polyclonal antibody, a multivalent antibody, a multispecific antibody (e.g., bispecific antibody), and/or an antibody fragment that binds to Bcl-xL. The antibody may be a chimeric antibody, a humanized antibody, a CDR-grafted antibody, or a human antibody, for example. The antibody fragment may be, for example, a Fab, Fab′, F(ab′)2, Fv, Fd, single chain Fv (scFv), disulfide bond Fv (sdFv), or a VL or a VH domain. The antibody may be in the form of a conjugate, for example, conjugated to a tag, a detectable label, or a cytotoxic agent. The antibody may be of the isotype IgG (e.g., IgG1, IgG2, IgG3 or IgG4), IgA, IgM, IgE or IgD.

Said oncolytic virus is at least one alphavirus; preferably, the alphavirus is at least one selected from the group consisting of M1 virus and Getah virus. The alphavirus (e.g., M1 virus, Getah virus) described herein can include these existing oncolytic viruses as well as those viruses that may have undergone natural variation, or mutation (natural or forced or selective), or genetic modification, or addition or deletion of or substitution of portion(s) sequences. The oncolytic virus described herein includes those viruses that have undergone the above-described changes.

Preferably said changes do not prevent said oncolytic viruses from exerting function recited in the present invention.

The present disclosure also provides a pharmaceutical composition for treating tumors, the composition comprising one or more Bcl-xL inhibitors and one or more oncolytic virus. The present disclosure also provides a pharmaceutical kit for treating tumors, comprising one or more Bcl-xL inhibitors and one or more oncolytic virus. The pharmaceutical kit differs from the composition in that in the pharmaceutical kit, the Bcl-xL inhibitor is not present in the same dosage form as the oncolytic virus, but is provided in a separate dosage form (such as one pill, or cascule or tablet or ampule comprising the Bcl-xL inhibitor(s) and another pill, or capsule or table or ampule comprising the oncolytic virus.) In some embodiments, a dosage form (oncolytic virus, Bcl-xL inhibitor or combination oncolytic virus and Bcl-xL inhibitor) can also contain one or more adjuvant. Said adjuvant could be such as aiding the therapeutic effect of a drug in pharmaceutical composition. The pharmaceutical kit can also contain a separately packaged Bcl-xL inhibitor and a separately packaged oncolytic virus. The Bcl-xL inhibitor and the oncolytic virus in the pharmaceutical kit can be administrated either simultaneously or sequentially in any order, such as where the Bcl-xL inhibitor is administered before the oncolytic virus, or the oncolytic virus is administered before the Bcl-xL inhibitor, or the Bcl-xL inhibitor and the oncolytic virus are administered together. In various embodiments, a patient can refer to a mammal. In some embodiments, the mammal can be a human.

Said Bcl-xL inhibitor includes but not limited to (S)-Gossypol acetic acid (Formula 1), Apogossypol (Formula 2), A-1155463 (Formula 3), AT-101 (R-(−)-gossypol acetic acid (Formula 4), WEHI-539 and WEHI-539 hydrochloride (Formula 5), Gambogic Acid (Formula 6), A-1210477 (Formula 7), ABT-263 (Formula 8), ABT-737 (Formula 9) and other compounds that inhibit the activity of Bcl-xL protein. Bcl-xL inhibitor also includes a tool for inhibiting the gene expression of Bcl-xL, including (but not limited to) RNA interference (RNAi), microRNA and gene editing or gene knockout material. The Bcl-xL inhibitor is preferably ABT-263, ABT-737 or a combination thereof.

Said oncolytic virus is at least one alphavirus; preferably, the alphavirus is at least one selected from the group consisting of M1 virus and Getah virus. In the composition or the pharmaceutical kit, ABT-263 or ABT-737 and the oncolytic virus are used in a ratio of optionally 0.01 to 15 mg:10³ to 10⁹ PFU; preferably 0.01 to 10 mg:10⁴ to 10⁹ PFU; more preferably 0.01 to 10 mg:10⁵ to 10⁹ PFU.

Preferably, the Bcl-xL inhibitor is used at a dosage ranging from 0.01 mg/kg to 15 mg/kg, while the oncolytic virus is used at a titer of MOI ranging from 10³ to 10⁹ (PFU/kg). In some embodiments, a dose providing an MOI of 10³ to 10⁴ or 10⁴ to 10⁵ or 10⁵ to 10⁶ or 10⁶ to 10⁷ or 10⁷ to 10⁸ or 10⁸ to 10⁹ PFU/kg can be used. In some embodiments, the Bcl-xL inhibitor is used at a dosage ranging from 0.01 mg/kg to 10 mg/kg, while the oncolytic virus is used at a titer of MOI ranging from 10⁴ to 10⁹ (PFU/kg); more preferably, the Bcl-xL inhibitor is used at a dosage ranging from 0.1 mg/kg to 10 mg/kg, while the oncolytic virus is used at a titer of MOI ranging from 10⁵ to 10⁹ (PFU/kg).

Preferably, ABT-263 or ABT-737 is used in a dosage ranging from 0.01 mg/kg to 15 mg/kg, while the oncolytic virus is used at a titer of MOI ranging from 10³ to 10⁹ (PFU/kg); preferably, ABT-263 or ABT-737 is used in a dosage ranging from 0.01 mg/kg to 10 mg/kg, while the oncolytic virus is used at a titer of MOI ranging from 10⁴ to 10⁹ (PFU/kg); more preferably, ABT-263 is used in a dosage ranging from 0.1 mg/kg to 10 mg/kg, while the oncolytic virus is used at a titer of MOI ranging from 10⁵ to 10⁹ (PFU/kg).

In an embodiment, said oncolytic virus is at least one alphavirus.

Preferably, the oncolytic virus is selected from the group consisting of M1 virus and Getah virus. M1 virus belongs to Getah-like virus and its homology with Getah virus is reported to be up to 97.8% in the relevant discovered viruses (Wen et al. Virus Genes. 2007; 35(3):597-603).

More information about M1 virus and Getah virus can also refer to Chinese Patent 104814984A.

The oncolytic virus described herein can include these existing oncolytic viruses as well as those viruses that may have undergone natural variation, or mutation (natural or forced or selective), or genetic modification, or addition or deletion of or substitution of portion(s) sequences. The oncolytic virus described herein includes those viruses that have undergone the above-described changes. Preferably said changes do not prevent said oncolytic viruses from exerting function recited in the present disclosure.

For example, the oncolytic virus may be the M1 virus as described in Genbank Accession No. EF011023, or may be a virus having a genome that is at least 80%, at least 85%, at least 90%, at least 91%, at least 92%, at least 93%, at least 94%, at least 95%, at least 96%, at least 97%, at least 98%, or at least 99%, identical to the genomic nucleotide sequence set forth in Genbank Accession No. EF011023.

In some embodiment, the oncolytic virus is M1 virus deposited with the China Center for Type Culture Collection on 17 Jul. 2014, and having a deposit number of CCTCC V201423. The oncolytic virus can also have a genome that is at least 80%, at least 85%, at least 90%, at least 91%, at least 92%, at least 93%, at least 94%, at least 95%, at least 96%, at least 97%, at least 98%, or at least 99%, identical to the genomic nucleotide of said M1 with a deposit number of CCTCC V201423.

Further, the oncolytic virus may be a Getah virus as described in Genbank Accession No. EU015062, or may be a virus having a genome that is at least 80%, at least 85%, at least 90%, at least 91%, at least 92%, at least 93%, at least 94%, at least 95%, at least 96%, at least 97%, at least 98%, or at least 99%, identical to the genomic nucleotide sequence set forth in Genbank Accession No. EU015062.

Optionally, a single oncolytic virus strain is used. In other embodiments, multiple strains and/or types of oncolytic virus are used.

In various embodiments, the combination of Bcl-xl inhibitor and oncolytic virus can be used to treat various types of tumors. Suitable tumors include solid tumors and blood tumors. In some embodiments, said solid tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer; preferably, said tumor is a tumor that is not sensitive to oncolytic virus; more preferably, said tumor is liver cancer, colorectal cancer, bladder cancer, breast cancer, cervical cancer, prostate cancer, glioma, melanoma pancreatic cancer, nasopharyngeal carcinoma, lung cancer, or gastric cancer, which is not sensitive to oncolytic virus. In a more preferred embodiment, the tumor is a tumor that is not sensitive to M1 oncolytic virus.

A single BcL-xL inhibitor may be used, or several may be used in combination, concurrently or in series. The Bcl-xL inhibitors, and/or the oncolytic virus, may be in the form of compositions comprising one or more inhibitors, and one or more carriers, excipients, diluents, pharmaceutically-acceptable carriers, stabilizers, buffering agents, preservatives, non-ionic detergents, antioxidants, and other additives. The pharmaceutical composition can be administered to a patient by a variety of routes such as orally, transdermally, subcutaneously, intranasally, intravenously, intramuscularly, intrathecally, topically or locally. Typically, the Bcl-xL inhibitors will be administered orally, parenterally, intravenously or subcutaneously. The Bcl-xL inhibitor may be present in a composition together with the oncolytic virus, or they may be present in separate compositions.

The present disclosure also relates to methods for treating tumors. In some embodiments, one or more Bcl-xL inhibitors and one or more oncolytic viruses are administered to a subject having a tumor. The tumor may be a solid tumor or a blood tumor. Preferably, the solid tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer; preferably, the tumor is a tumor that is not sensitive to oncolytic virus; more preferably, the tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer, which is not sensitive to oncolytic virus. The Bcl-xL inhibitor may be administered concurrently, before, or subsequent to, administration of an oncolytic virus contemplated herein. Additionally, the Bcl-xL inhibitor and/or the oncolytic virus may be administered once a week, or several times (e.g., 2, 3, 4, 5, 6, 7, 8, 9 or 10) a week. The Bcl-xL inhibitor and/or the oncolytic virus may be administered for one or several weeks (1, 2, 3, 4, 5, 6, 7, 8, 9, or 10), for a month, or even for several months (2, 3, 4, 5, 6, 7, 8, 9, 10, 11 or 12 or more).

As some embodiments, Bcl-xl inhibitors (such as ABT-263, ABT-737 or a combination thereof) can be administered by injection or in the form of a tablet, capsule, patch, kit, etc. In some embodiments, Bcl-xl inhibitors can be part of a kit. In some embodiments, the combination of Bcl-xl inhibitor and oncolytic virus can be administered by injection; preferably, by intravenous injection.

The present disclosure shows that Bcl-xL inhibitor, such as ABT-263 or ABT-737, can enhance the anti-tumor effect of oncolytic virus so as to enhance the therapeutic efficacy of the oncolytic virus serving as an antitumor drug. Cytological experiments demonstrated that the combination of M1 virus and Bcl-xL inhibitor (such as ABT-263 or ABT-737) can significantly cause pathological change of the tumor cells in terms of morphology, which significantly inhibit tumor cell growth.

In some embodiments, Bcl-xl inhibitors (ABT-263 or ABT-737) was used in combination with M1 virus to treat human liver cancer cell strain Hep3B. Surprisingly, it is found that a combination of the antiviral compounds ABT-263 or ABT-737 and M1 virus significantly increased pathological change of the tumor cells in terms of morphology, and significantly decreased the survival rate of the tumor cells. In some cases, the morphological change in tumor cells included cell swelling, nuclear condensation and fragmentation with cells then undergoing apoptosis. For example, in one embodiment, the liver cancer cells treated with M1 virus (MOI=0.001) alone (without the use of a Bcl-xl inhibitor) had a survival rate of 81.5%, whereas the tumor cells treated with the combination of 100 nM ABT-263 or ABT-737 and M1 virus at the same MOI had a survival rate dramatically reduced to 25.2%. Evidently, compared with the anti-tumor effect obtained from the treatment with M1 virus alone, a combination use of Bcl-xl inhibitors (such as ABT-263) and M1 produced significantly enhanced oncolytic effect.

Chinese patent CN 201510990705.7 previously submitted by the inventor discloses that chrysophanol and its derivatives are used as anti-tumor synergists for M1 virus, and a combination of 50 μM chrysophanol and M1 virus (MOI=0.001) could reduce the survival rate of tumor cells to 39.6%. However, it can be desirable to achieve higher anti-tumor strength of virus-synergists combination and/or to achieve lower or no effect on normal cells. The present disclosure shows that the combination of Bcl-xl inhibitor(s) and oncolytic virus significantly reduced the survival rate of tumor cells. For example, a combination use of 100 nM ABT-263 and M1 virus significantly reduced the survival rate of tumor cells to 25.2%. Compared with chrysophanol and its derivatives thereof, the present disclosed anti-tumor synergist for M1 virus could significantly increased the killing effect on tumor cells. In addition, the Bcl-xl inhibitor (e.g., ABT-263) was used in a pharmaceutically effect dose that was merely 2/1000 of chrysophanol, acting quickly as anti-tumor synergist. Time required for Bcl-xl inhibitor (e.g., ABT-263) treatment is only ⅔ of those needed for chrysophanol (72 hs needed for treatment with chrysophanol vs. 48 hs needed for treatment with ABT-263).

It was reported that Bcl-2 family inhibitors such as ABT-263, ABT-737 and ABT-199 have an anti-tumor effect by inhibiting the anti-apoptotic proteins Bcl2, Bcl-xL and Bcl-w in tumor cells. However, as presented herein, not all of the Bcl-2 family inhibitors can synergistically enhance the oncolytic effect of oncolytic virus. It is unexpectable that the Bcl-xL inhibitor can synergistically enhance the anti-tumor effect of oncolytic virus, whereas neither Bcl-2 inhibitor nor Bcl-w inhibitor synergistically enhances the oncolytic effect of oncolytic virus.

As presented hererin, when Bcl-xL inhibitor (e.g., ABT-263 or ABT-737) is used in combination with an oncolytic virus to treat tumor cells, the killing effect on the tumor cells is higher than that obtained from the use of Bcl-xL inhibitor (e.g., ABT-263 or ABT-737) alone when used at the same concentration. For example, when tumor cells were treated with 100 nM ABT-263, the tumor cells had a survival rate of up to 88.8%, and when tumor cells were treated with 100 nM ABT-263 in combination with M1 virus, the tumor cells had a survival rate that is sharply reduced to 25.2%. Accordingly, greatly increased oncolytic effect was produced by the combination of ABT-263 and M1 by the synergistic action mechanism between ABT-263 and M1 virus, rather than the simple anti-tumor mechanism of ABT-263.

Specific Mode for Carrying Out the Invention

The present invention is further illustrated by the following examples. Nevertheless, the embodiments of the present invention are not limited to the following description of the examples. Any equivalent changes or modifications made according to the principle or spirit of the present invention should be deemed as fall within the protection scope of the present invention.

The materials and experiment methods used in the present invention are conventional materials and methods, unless otherwise specified.

### Example 1. ABT-263 and M1 Virus Significantly Increase the Pathological Change of Human Liver Cancer Cell Strain in Terms of Morphology

Materials:

Human liver cancer cell Hep3B (purchased from ATCC), M1 virus (obtained with CCTCC V201423), a high glucose-containing DMEM culture medium (4.5 g/l glucose) (purchased from Corning), and an inverted phase contrast microscope.

Methods:

a) Cultivation of cells: human liver cancer cell Hep3B was grown in a DMEM complete culture medium containing 10% FBS, 100 U/ml penicillin and 0.1 mg/ml streptomycin; all the cell strains were placed in a closed incubator with 5% CO₂ at 37° C. constant temperature (relative humidity 95%) for subculture. Growth of the cell strains was observed by the inverted microscope. Cells are passaged about every 2 to 3 days, and the cells in exponential growth phase were taken for a formal experiment.
b) Cell treatment and morphological observation: the cells in exponential growth phase were selected and added into a DMEM complete culture medium (containing 10% fetal bovine serum, 1% penicillin/streptomycin (Life Technologies)) to prepare a cell suspension. The cells were inoculated into a 24-well culture plate at a density of 2.5×10⁴/well. 48 hours after the treatment with ABT-263 (100 nM) alone, the infection with M1 (MOI=0.001) alone, and the combined treatment with M1 (MOI=0.001) and ABT-263 (100 nM), the morphological change of cells was observed by the inverted phase contrast microscope, using a group that was not treated with M1 virus and ABT-263 as the control group.

Results:

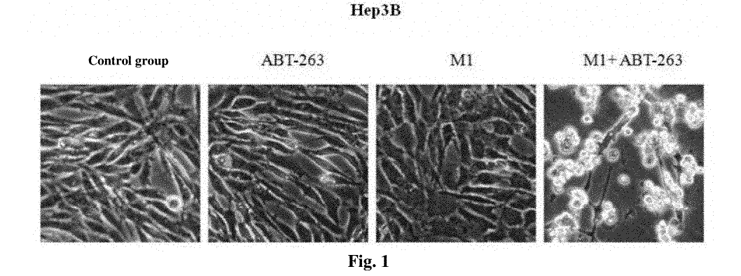

As shown in FIG. 1, the morphology of the cells was observed by the phase-contrast microscope. Hep3B cells were grown in adherent monoculture, and the cells were closely arranged with the uniform phenotype. 48 hours after the treatment with ABT-263 (100 nM) and M1 virus (MOI=0.001), the morphology of the cells was significantly changed. Compared with the cells in the control group, the group treated with M1 alone and the group treated with ABT-263 alone, the number of the viable cells in the combined treatment group was significantly reduced, and the morphology of the viable cells was significantly changed with their cell bodies. contracted to a spherical shape, and their refractive index was significantly increased. A pathological change of death was shown.

### Example 2. Combined Treatment of ABT-263 or ABT-737 and M1 Virus Significantly Reduces the Survival Rate of Human Cancer Cell Strain

Materials:

Human liver cancer cell Hep3B, human bladder cancer cell T24, human colorectal cancer cell LoVo, M1 virus, a high glucose-containing DMEM culture medium, and an automatic microplate reader for enzyme-linked immunosorbent assay. Cells and virus, are from the same sources as Example 1.

Methods:

a) Inoculation of cells and administration treatment: The cells in the exponential growth phase were selected and added into a DMEM complete culture medium (containing 10% fetal bovine serum and 1% penicillin/streptomycin (Life Technologies)) to prepare a cell suspension. The cells were inoculated into a 96-well culture plate at a density of 4×10³/well. After 12 hours, the cells were completely adherent to the wall. The wells were divided into a control group, a group treated with ABT-263 alone, a group infected with M1 alone, a group treated with the combination of ABT-263 and M1 and a group treated with the combination of ABT-737 and M1. The dose was used as follows: M1 virus that infected the cells; different dose gradient set for M1 virus.
b) Reaction of MTT (3-(4,5-dimethylthiazol-2-yl)-2,5-diphenyltetrazolium bromide or methyl thiazolyl tetrazolium) with succinate dehydrogenase in the cells: 48 hours after culture, 20 μl of MIT (5 mg/ml) was added into each well, and was incubated for further 4 hours. After incubation, granular blue and purple formazan crystals formed in the living cells were observed by microscopic examination.
c) Dissolution of formazan particles formed in the cells was accomplished as follows: the supernatant was carefully sucked off, and 100 μl/well of DMSO was added to dissolve the formed crystals, and was shaken in a microoscillator for 5 minutes. Then, the optical density (OD value) of each well was detected in an enzyme linked detector at a wavelength of 570 nm. The experiments were repeated for 3 times for each group. The survival rate of the cells=OD value of the drug treatment group/OD value of the control group×100%.

Results:

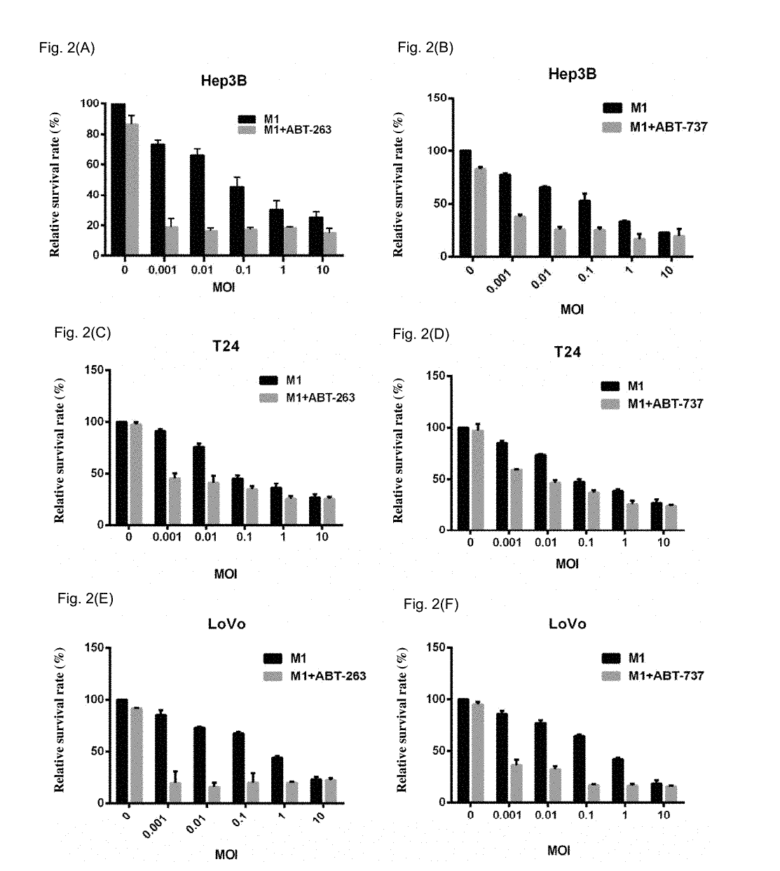

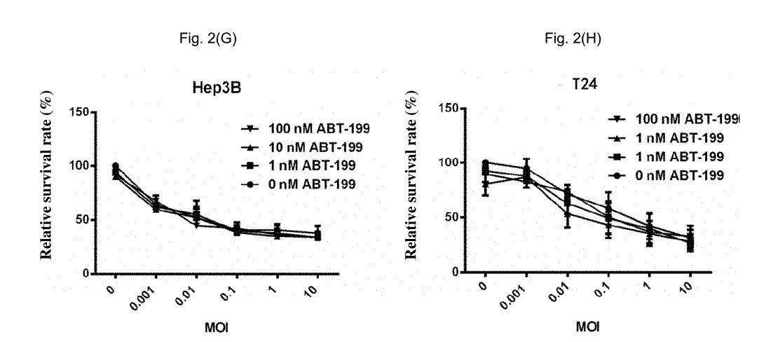

As shown in FIG. 2a, treatment with M1 virus alone result in a relatively lower inhibition efficacy on tumor cell Hep3B (the survival rate of the tumor cells reaches 81.5%), and the tumor cells treated with 100 nM ABT-263 still had a survival rate of up to 88.8%. However, when the tumor cells were treated with the combination of 100 nM ABT-263 and M1 virus having the same MOI (ABT-263+M1), the survival rate was greatly reduced to 25.2%. Likewise, compared with the group treated with M1 virus alone and the group treated with ABT-263 alone, when tumor cells were treated with the combination of ABT-263 and M1 at various doses, the survival rate was greatly reduced. Similar results were observed for Hep3B in the group treated with the combination of ABT-739 and M1 (FIG. 2b). In addition, the combination of the Bcl-xL inhibitor ABT-737 and M1 also significantly reduced the survival rate of tumor cells T24 (FIGS. 2c and d) and LoVo (FIGS. 2e and 2f).

However, when comparing with the group treated with M1 virus alone and the group treated with the Bcl-2 selective inhibitor ABT-199 alone, tumor cells treated with the combination of ABT-199 and M1 at various doses did not show significant difference in the survival rate. It is shown that inhibition of Bcl-2 cannot result in an increased oncolytic effect of M1 virus (FIGS. 2g and 2h).

### Example 3. Inhibition of Bcl-xL in Combination with the Application of M1 Oncolytic Virus Achieved Anti-Tumor Effect

Materials:

M1 virus as described in example 1, human liver cancer cell Hep3B, bladder cancer cell T24, RNA interference fragments of Bcl-xL and Bcl-w, MTT (methyl thiazolyl tetrazolium) (purchased from MPbio) and a phase contrast microscope. Cells and virus are from the same sources as Example 1.

interference fragment (SiRNA) of Bcl-xL:
sense strand
(SEQ ID NO. 1)
5′-GGAUACAGCUGGAGUCAGUdTdT-3′
antisense strand
(SEQ ID NO. 2)
5′-ACUGACUCCAGCUGUAUCCdTdT-3′
interference fragment (SiRNA) of Bcl-w:
sense strand
(SEQ ID NO. 3)
5′-CAGCUGUAUUCCAUUACAUdTdT-3′
antisense strand
(SEQ ID NO. 4)
5′-AUCUAAUGGAAUACAGCUGdTdT-3′

Methods:

The cells in exponential growth phase were selected and added into a DMEM complete culture medium to prepare a cell suspension. The cells were inoculated in a 6-well plate at a density of 1×10⁵/well. After 24 hours, SiRNA target gene fragment wrapped with liposomes was added. After 24 hours, the cells were infected with M1 virus. 48 hours after infection, the samples were treated.

(1) The morphological change was observed by the phase contrast microscope;
(2) The protein samples were collected, and the interference efficiency and the M1 virus proteins NS3 and E1 were detected by Western blot.
(3) The survival rate of the cells was calculated by MTT method.

Results:

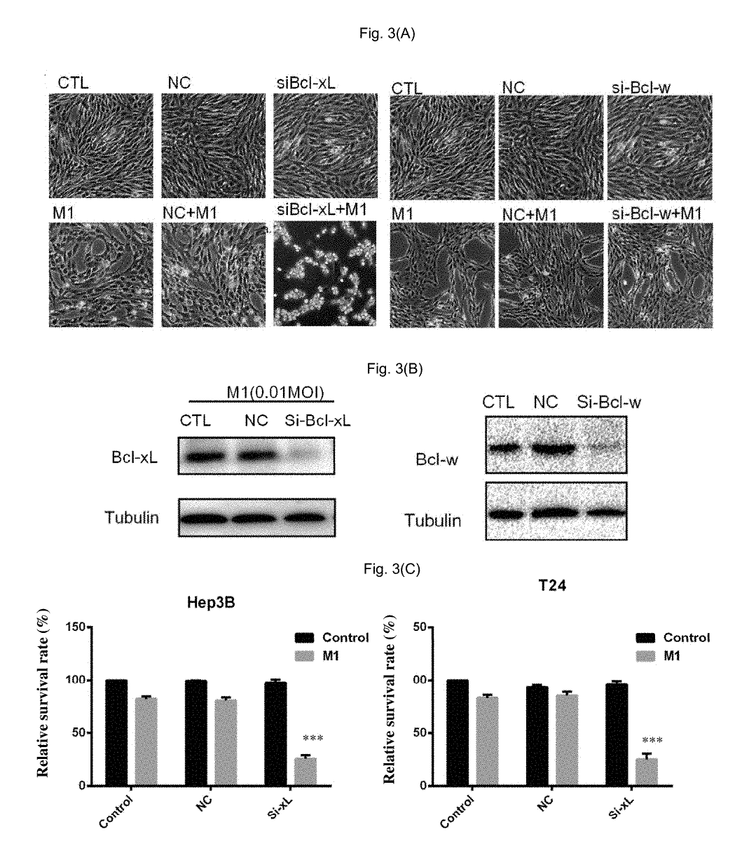

After interfering Bcl-xL and Bcl-w respectively, it was found in the Western blot detection result that the gene expressions of Bcl-xL and Bcl-w (FIG. 3b) were significantly decreased. When compared with the treatment of non-interference group (CTL) or the garbled interference group (NC), the treatment with Bcl-xL interference or Bcl-w interference alone did not result in pathological change to cell morphology. Also, use of M1 virus alone did not cause pathological change of cell morphology. However, a combination of interference of Bcl-xL and the application of M1 rivus (siBc1-xL+M1) caused significant pathological change of the cell morphology. On the other hand, a combination of interference of Bcl-w and the application of M1 rivus (siBc1-w+M1) could not cause pathological change of the cell morphology (FIG. 3a). MTT assays (FIG. 3c) with the results analyzed by ANOVA with *** representing p<0.001, showed that among those tested Bcl-2 members, only a combination of interference of Bcl-xl and the application of M1 rivus caused significant decease in the survival rate of the human liver cancer cell Hep3B and bladder cancer cell T24.

The above results showed that the oncolytic effect of M1 can be enhanced by inhibiting Bcl-xL. However, inhibition of Bcl-w cannot synergistically enhance the oncolytic effect of M1.

### Example 4. The Combination of ABT-263 and M1 Virus Significantly Inhibits the Growth of Transplantable Tumor of Human Liver Cancer Cell Strain

Materials:

M1 virus as described in example 1, liver cancer cell strain Hep3B, colorectal cancer cell strain LoVo, and 4-week-aged female BALB/c nude mice (Model animal research center of Nanjing University). Cells and virus are from the same sources as Example 1.

Methods:

This experiment was designed in a randomized, single-blind way. 5×10⁶ Hep3B or LoVo cells were subcutaneously injected into dorsa of the 4-week-aged BALB/c nude mice.

When the size of the tumor reached 50 mm³, the mice were divided into groups, including an untreated control group, a group treated with ABT-263 alone (i.p. 10 mg/kg/d), a group infected with M1 alone (tail vein injection with M1 virus of 2×10⁶ PFU/time), and a group treated with a combination of ABT-263 and M1 (ABT-263 and M1 virus were administered in the same way at the same dose); and three injections were performed continuously (Three injections was performed on days 6-8). The weight, length and width of the tumor were measured every two days, and the volume of the tumor was calculated according to the formula: (length×width 2)/2. After the volume of the tumor was measured, One-way ANOVA statistics was carried out, wherein *** represented p<0.001 and **represented p<0.01.

Results:

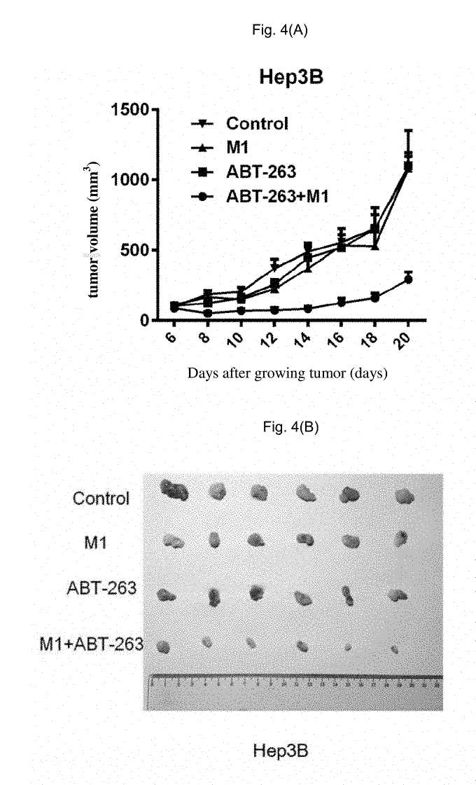

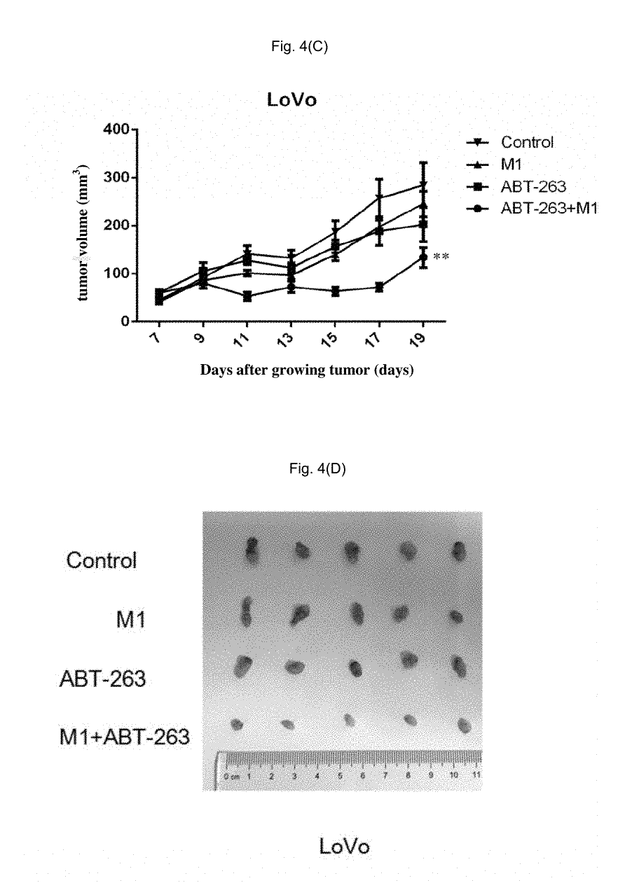

Pathological anatomy was carried out in the animals with transplantable tumors of two tumor cells to measure the volume of the tumors. The results showed that compared with the control group, the group treated with ABT-263 alone and the group infected with M1 alone only showed slight shrinkage of the tumor volume, while the group treated with the combination of ABT-263 and M1 showed significant shrinkage of the tumor volume (FIGS. 4b and 4d), and the one way ANOVA indicated that there was a statistical difference (FIGS. 4a and 4c). This synergistic effect was significantly enhanced especially in tumors that were less sensitive to a single drug, and the tumor volume was reduced by a surprising degree (FIG. 4a).

The above-described examples are merely illustrative embodiments of the present invention. Nevertheless, the embodiments of the present invention are not limited to the above-described examples. Any other change, modification, replacement, combination, and simplification without departing from the spirit and principle of the present invention are all included in the protection scope of the present invention.

1. Use of Bcl-xL inhibitor in the preparation of an anti-tumor synergist for oncolytic virus, preferably, said oncolytic virus is at least one alphavirus.

2. The use of claim 1, wherein said alphavirus is at least one selected from the group consisting of M1 virus and Getah virus;
more preferably, said alphavirus has at least 97.8% nucleotide sequence identity to the genome of the M1 virus deposited under Accession No. CCTCC V201423; more preferably, said alphavirus has at least 100% nucleotide sequence identity to the genome of the M1 virus deposited under Accession No. CCTCC V201423;
preferably, said Bcl-xL inhibitor is a substance that inhibits the activity of Bcl-xL protein, a substance that degrades Bcl-xL protein, or a genetic tool that reduces the level of Bcl-xL protein;
preferably, said substance that inhibits the activity of Bcl-xL protein is selected from a compound, more preferably, said compound is one or more selected from the group consisting of (S)-Gossypol acetic acid, Apogossypol, A-1155463, AT-101, WEHI-539, WEHI-539 hydrochloride, Gambogic Acid, A-1210477, ABT-263 and ABT-737;
or preferably, said genetic tool that reduces the level of Bcl-xL protein is RNA interference, microRNA or gene editing or gene knockout material.

3. A kit or a pharmaceutical composition, comprising:
(a) at least one Bcl-xL inhibitor; and
(b) at least one oncolytic virus;
preferably, said composition is a composition for treating tumors.

4. The kit of claim 3, wherein the at least one Bcl-xL inhibitor and the at least one oncolytic virus are present as separate components.

5. (canceled)

6. The composition/kit according to claim 3, wherein the Bcl-xL protein inhibitor is ABT-263, ABT-737 or a combination thereof;

7. The composition/kit according to claim 3, preferably, said at least one oncolytic virus is M1 virus, Getah virus or a combination thereof;
more preferably, said alphavirus has at least 97.8% nucleotide sequence identity to the genome of the M1 virus deposited under Accession No. CCTCC V201423; more preferably, said alphavirus has at least 100% nucleotide sequence identity to the genome of the M1 virus deposited under Accession No. CCTCC V201423.

8. The composition/kit according claim 3, wherein the tumor is a solid tumor or a blood tumor;
preferably, the solid tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer;
or preferably, the tumor is a tumor that is not sensitive to oncolytic virus;
more preferably, the tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer, which is not sensitive to oncolytic virus.

9. The composition/pharmaceutical kit according to claim 3, wherein said Bcl-xL inhibitor and the oncolytic virus are used in a ratio of 0.01 to 15 mg:10³ to 10⁹ PFU; preferably 0.01 to 10 mg: 10⁴ to 10⁹ PFU; more preferably 0.01 to 10 mg:10⁵ to 10⁹ PFU;
more preferably, said Bcl-xL inhibitor is used in a dosage ranging from 0.01 mg/kg to 15 mg/kg while the oncolytic virus is used at a titer of MOI ranging from 10³ to 10⁹ (PFU/kg); preferably, said Bcl-xL inhibitor is used in a dosage ranging from 0.01 mg/kg to 10 mg/kg, while the oncolytic virus is used at a titer of MOI ranging from 10⁴ to 10⁹ (PFU/kg); more preferably, said Bcl-xL inhibitor is used in a dosage ranging 0.1 mg/kg to 10 mg/Kg, while the oncolytic virus is used at a titer of MOI ranging from 10⁵ to 10⁹ (PFU/kg);
more preferably, said Bcl-xL inhibitor is ABT-263, ABT-737 or a combination thereof; more preferably, said Bcl-xL inhibitor is ABT-263 or ABT-737.

10. The pharmaceutical composition according to claim 3, wherein the composition further comprises a pharmaceutically acceptable carrier; preferably said composition is in the form of a lyophilized powder, injection, tablet, capsule, kit or patch.

11. A method for treating a subject with a tumor, said method comprising administering to the subject at least one Bcl-xL inhibitor and at least one oncolytic virus.

12. The method of claim 11, wherein said at least one Bcl-xL inhibitor is a substance that inhibits Bcl-xL;
preferably, said at least one Bcl-xL inhibitor is a substance inhibits the activity of Bcl-xL protein, a substance that degrades Bcl-xL protein, or a genetic tool that reduces the level of Bcl-xL protein.

13. The method of claim 11, wherein said oncolytic virus include at least one alphavirus.

14. The method of claim 11, wherein the tumor is a solid tumor or a blood tumor.

15. The method of claim 11, wherein the at least one Bcl-xL inhibitor and the at least one oncolytic virus are administered separately.

16. The kit or the pharmaceutical composition according to claim 3, wherein said at least one Bcl-xL inhibitor is a substance that inhibits Bcl-xL; preferably, said at least one Bcl-xL inhibitor is a substance inhibits the activity of Bcl-xL protein, a substance that degrades Bcl-xL protein, or a genetic tool that reduces the level of Bcl-xL protein;
preferably, said at least one Bcl-xL inhibitor is a low molecular weight compound;
preferably, said substance that inhibits the activity of Bcl-xL protein is selected from a compound;
more preferably, the compound is one or more selected from the group consisting of (S)-Gossypol acetic acid, Apogossypol, A-1155463, AT-101, WEHI-539, WEHI-539 hydrochloride, Gambogic Acid, A-1210477, ABT-263 and ABT-737;
or preferably, said genetic tool that reduces the level of Bcl-xL protein is RNA interference, microRNA or gene editing or gene knockout material;

17. The kit or the pharmaceutical composition according to claim 3, wherein said oncolytic virus is at least one alphavirus;
preferably, the alphavirus is an M1 virus or a Getah virus;
more preferably, said alphavirus has at least 97.8% nucleotide sequence identity to the genome of the M1 virus deposited under Accession No. CCTCC V201423; more preferably, said alphavirus has at least 100% nucleotide sequence identity to the genome of the M1 virus deposited under Accession No. CCTCC V201423.

18. The method of claim 12, wherein said at least one Bcl-xL inhibitor is a low molecular weight compound;
preferably, said substance that inhibits the activity of Bcl-xL protein is selected from a compound;
more preferably, the compound is one or more selected from the group consisting of (S)-Gossypol acetic acid, Apogossypol, A-1155463, AT-101, WEHI-539, WEHI-539 hydrochloride, Gambogic Acid, A-1210477, ABT-263 and ABT-737;
or preferably, said genetic tool that reduces the level of Bcl-xL protein is RNA interference, microRNA or gene editing or gene knockout material.

19. The method of claim 12, wherein said Bcl-xL inhibitor is an siRNA, an shRNA, or an antibody;
preferably, the Bcl-xL inhibitor is a nucleic acid that is at least 90% at least 91%, at least 92%, at least 93%, at least 94%, at least 95%, at least 96%, at least 97%, at least 98%, or at least 99% identical to any of the nucleic acids set forth as SEQ ID Nos 1-4.

20. The method of claim 13, wherein the alphavirus is an M1 virus or a Getah virus;
optionally, said M1 virus have a genome as described in Genbank Accession No. EF011023, or have a genome that is at least 80%, at least 85%, at least 90%, at least 91%, at least 92%, at least 93%, at least 94%, at least 95%, at least 96%, at least 97%, at least 98%, or at least 99%, identical to the genomic nucleotide sequence set forth in Genbank Accession No. EF011023;
optionally, said Getah virus have a genome as described in Genbank Accession No. EU015062, or have a genome that is at least 80%, at least 85%, at least 90%, at least 91%, at least 92%, at least 93%, at least 94%, at least 95%, at least 96%, at least 97%, at least 98%, or at least 99%, identical to the genomic nucleotide sequence set forth in Genbank Accession No. EU015062;
or optionally, said M1 virus has at least 80%, at least 85%, at least 90%, at least 91%, at least 92%, at least 93%, at least 94%, at least 95%, at least 96%, at least 97%, at least 98%, or at least 99% nucleotide sequence identity to the genome of the M1 virus deposited under Accession No. CCTCC V201423;
or optionally, said alphavirus has at least 97.8% nucleotide sequence identity to the genome of the M1 virus deposited under Accession No. CCTCC V201423; more preferably, said alphavirus has at least 100% nucleotide sequence identity to the genome of the M1 virus deposited under Accession No. CCTCC V201423.

21. The method of claim 14, wherein the solid tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer; or
the tumor is a tumor that is not sensitive to oncolytic virus;
preferably, the tumor is liver cancer, colorectal cancer, bladder cancer, breast cancer, cervical cancer, prostate cancer, glioma, melanoma, pancreatic cancer, nasopharyngeal carcinoma, lung cancer, or gastric cancer, which is not sensitive to oncolytic virus.
